(19) United States
(12) Patent Application Publication
(10) Pub. No.: US 2017/0043214 A1
(43) Pub. Date: Feb. 16, 2017

(54) PHYSICAL ACTIVITY ASSISTANCE APPARATUS, PHYSICAL ACTIVITY ASSISTANCE SYSTEM, PHYSICAL ACTIVITY ASSISTANCE METHOD, AND PHYSICAL ACTIVITY ASSISTANCE PROGRAM

(71) Applicant: Seiko Epson Corporation, Tokyo (JP)

(72) Inventor: Daisuke Higashi, Suwa-shi (JP)

(21) Appl. No.: 15/232,225

(22) Filed: Aug. 9, 2016

(30) Foreign Application Priority Data
Aug. 11, 2015 (JP) 2015-158980

(51) Int. Cl. A63B 24/00; A61B 5/00; A61B 5/113; A61B 5/1455; A61B 5/08

(52) U.S. Cl. CPC A63B 24/0062; A61B 5/14552; A61B 5/08; A61B 5/1135; A61B 5/746; A61B 5/0022; A61B 2560/0247; A63B 2024/0068; A63B 2230/207; A63B 2230/40; A63B 2220/836; A63B 2220/73

(57) ABSTRACT

A physical activity assistance apparatus includes a blood oxygen concentration measurement section (biological information detection section and blood oxygen concentration calculation section) that measures a user's blood oxygen concentration and a breathing instruction section that instructs the user to practice a predetermined breathing method when the blood oxygen concentration is lower than or equal to a first threshold. The thus configured physical activity assistance apparatus can appropriately support the user's physical activity.

### CROSS-REFERENCES TO RELATED APPLICATIONS

This application claims priority to Japanese Patent Application No. 2015-158980, filed Aug. 11, 2015, the entirety of which is herein incorporated by reference.

### BACKGROUND

1. Technical Field

The present invention relates to a physical activity assistance apparatus, a physical activity assistance system, a physical activity assistance method, and a physical activity assistance program.

2. Related Art

There has been a known travel assistance information providing system of related art that generates travel assistance information in consideration of a user's physical condition (see JP-A-2013-220182, for example).

The travel assistance information providing system described in JP-A-2013-220182 includes an environment prediction section and a travel assistance information generation section. Out of the two sections, the environment prediction section uses environment information (atmospheric pressure, temperature, humidity, and wind direction, for example) along a path along which the user travels to predict changes in the environment along the path. The travel assistance information generation section uses a result of the predication performed by the environment prediction section and the user's biological information (heart rate, pulse rate, and blood sugar level, for example) to generate travel assistance information including advice on the user's travel. The user then acts on the basis of the travel assistance information to avoid a situation in which the user is involved in a risky state.

In the travel assistance information providing system described in JP-A-2013-220182, in which the travel assistance information is provided on the basis of the user's biological information and the environment information along a path along which the user travels, when the user is, for example, climbing a mountain, the system abruptly recommends the user to descend the mountain on the basis of the information described above. For example, even in a case where the user's condition, such as the degree of altitude sickness, is likely to be improved by causing the user to take a rest for a while or practice a predetermined breathing method, that is, even in a case where the user does not necessarily descend the mountain, the system recommends the user to descend the mountain in some cases. The user cannot therefore continue physical activity (mountain climbing described above, for example) that the user can potentially perform in some cases, and the system undesirably cannot always appropriately support the user's physical activity.

### SUMMARY

An advantage of some aspects of the invention is to provide a physical activity assistance apparatus, a physical activity assistance system, a physical activity assistance method, and a physical activity assistance program capable of appropriately supporting a user's physical activity.

A physical activity assistance apparatus according to a first aspect of the invention includes a blood oxygen concentration measurement section that measures a user's blood oxygen concentration and a breathing instruction section that instructs the user to practice a predetermined breathing method when the blood oxygen concentration is lower than or equal to a first threshold.

A pressure breathing method can be exemplified as the predetermined breathing method described above. In the first aspect, for example, in a case where the first threshold is set at a value below which the user is likely to suffer from altitude sickness, the user is instructed, when the blood oxygen concentration is lower than or equal to the first threshold, to practice a breathing method that allows the blood oxygen concentration to rise, whereby the user can lower a risk of developing altitude sickness by practicing breathing in accordance with the instruction. Therefore, for example, when the user practices the breathing method to successfully cause the blood oxygen concentration to be higher than the first threshold, the user can continue the physical activity. The physical activity assistance apparatus can therefore appropriately support the user's physical activity.

In the first aspect, it is preferable that the physical activity assistance apparatus preferably further include a breathing detection section that detects the user's breathing, a practice evaluation section that evaluates based on a result of the detection performed by the breathing detection section whether or not the user is practicing breathing according to the breathing method, and a handling method instruction section that instructs the user to practice a handling method when the practice evaluation section determines that the user is practicing the breathing according to the breathing method and the measured blood oxygen concentration is lower than or equal to a second threshold lower than the first threshold.

Suspension of the physical activity can be exemplified as the handling method described above. For example, when the user is climbing a mountain, descending the mountain can be exemplified as the handling method.

According to the first aspect with the configuration described above, in a case where the blood oxygen concentration is lower than or equal to the second threshold, which is lower than the first threshold, even after the user practices the breathing method, the user is instructed to practice the handling method, whereby a situation in which the user continues the physical activity and is involved in a risky state (altitude sickness, for example) can be avoided. The physical activity assistance apparatus can therefore appropriately support the user's physical activity.

In the first aspect, it is preferable that the physical activity assistance apparatus further includes a breathing detection section that detects the user' s breathing, a practice evaluation section that evaluates based on a result of the detection performed by the breathing detection section whether or not the user is practicing breathing according to the breathing method, a period acquisition section that acquires, when the practice evaluation section determines that the user is practicing the breathing according to the breathing method, a period for which the user has been practicing the breathing according to the breathing method, and a handling method instruction section that instructs the user to practice a handling method when the acquired period is longer than a predetermined period and the measured blood oxygen concentration is not higher than a second threshold lower than the first threshold.

According to the first aspect with the configuration described above, in a case where the blood oxygen concentration is not higher than the second threshold even after the user practices the breathing method for the predetermined period, the user is instructed to practice the handling method, whereby a situation in which the user continues the physical activity and is involved in a risky state can be further avoided. The physical activity assistance apparatus can therefore more appropriately support the user's physical activity.

In the first aspect, it is preferable that the breathing detection section includes a first sensor that detects motion of the user's chest.

According to the first aspect with the configuration described above, since the first sensor can detect the motion of the user's chest, the user's breathing rate can be readily measured. The practice evaluation section can therefore evaluate on the basis of the breathing rate whether or not the user is appropriately practicing the breathing method. The physical activity assistance apparatus can therefore more adequately provide the user with a handling method instruction.

In the first aspect, it is preferable that the breathing detection section includes a second sensor that detects the user's body motion, and that the practice evaluation section evaluates based on results of the detection performed with the first sensor and the second sensor whether or not the user is practicing the breathing according to the breathing method.

According to the first aspect with the configuration described above, since the breathing evaluation section evaluates whether or not the user is practicing the breathing method on the basis of the motion of the user's body motion and chest motion, the breathing evaluation section can more accurately evaluate whether or not the user is practicing the breathing method. The physical activity assistance apparatus can therefore further adequately provide the user with a handling method instruction.

In the first aspect, it is preferable that the physical activity assistance apparatus further includes an altitude detection section that detects altitude and an adjustment section that adjusts the first threshold in accordance with the detected altitude.

A GPS (global positioning system), an atmospheric pressure meter, and other devices can be exemplified as the altitude detection section.

In the first aspect with the configuration described above, in the case where the first threshold is set at a value below which the user is likely to suffer from altitude sickness, the first threshold can be adjusted on an altitude basis. The physical activity assistance apparatus can therefore adequately provide the user with a handling method instruction in accordance with the altitude.

In the first aspect, it is preferable that the physical activity assistance apparatus further includes a restriction section that restricts an instruction issued by the breathing instruction section when the blood oxygen concentration is higher than the first threshold.

According to the first aspect with the configuration described above, since the instruction issued by the breathing instruction section is restricted when the blood oxygen concentration is higher than the first threshold, that is, when the user's blood oxygen concentration reaches an appropriate value, the user can recognize that the blood oxygen concentration has reached the appropriate value. The fact the instruction is no longer made therefore allows the user to readily recognize that the user can continue the physical activity (mountain climbing, for example).

A physical activity assistance system according to a second aspect of the invention includes the physical activity assistance apparatus and an information processing apparatus capable of communicating with the physical activity assistance apparatus. The physical activity assistance apparatus transmits a warning signal when the blood oxygen concentration is lower than or equal to the first threshold and the second threshold, and the information processing apparatus includes a notification section that notifies the user of a warning when the warning signal is received.

The second aspect can provide the same advantageous effects as those provided by the physical activity assistance apparatus according to the first aspect. Further, a leader of a mountain climbing party or any other person who possesses the information processing apparatus described above can grasp both a state in which a member of the party is likely to suffer from altitude sickness and a state in which the member has suffered from altitude sickness.

A physical activity assistance method according to a third aspect of the invention is a physical activity assistance method performed with use of an instruction apparatus that instructs a user to practice a breathing method, the method including measuring the user's blood oxygen concentration and instructing the user to practice a predetermined breathing method when the blood oxygen concentration is lower than or equal to a first threshold.

The third aspect can provide the same advantageous effects as those provided by the physical activity assistance apparatus according to the first aspect described above.

A physical activity assistance program according to a fourth aspect of the invention is a physical activity assistance program executed by a computer, the program causing the computer to perform acquiring a user's blood oxygen concentration and instructing the user to practice a predetermined breathing method when the blood oxygen concentration is lower than or equal to a first threshold.

The fourth aspect, in which the computer executes the physical activity assistance program, can provide the same advantageous effects as those provided by the physical activity assistance apparatus according to the first aspect described above.

The physical activity assistance program may be recorded on a computer readable recording medium.

The configuration described above, in which the computer reads and executes as appropriate the physical activity assistance program recorded on the recording medium, can provide the same advantageous effects as those provided by the physical activity assistance apparatus according to the first aspect described above. As the recording medium, a magnetic tape, a magnetic disk, an optical disk, a magneto-optical disk, an HDD (hard disk drive), and a semiconductor memory can be employed. The physical activity assistance program can be not only installed in the computer and executed but also readily distributed by using any of the recording media described above. Further, the physical activity assistance program may be acquired from a server or any other apparatus on a network, installed in a computer and executably distributed.

### BRIEF DESCRIPTION OF THE DRAWINGS

The invention will be described with reference to the accompanying drawings, wherein like numbers reference like elements.

FIG. 1 is a diagrammatic view showing the configuration of a physical activity assistance system according to a first embodiment of the invention.

FIG. 2 is a block diagram showing the configuration of a biological information measurement apparatus that forms the physical activity assistance system according to the first embodiment.

FIG. 3 is a block diagram showing the configuration of a control section of the biological information measurement apparatus according to the first embodiment.

FIG. 4 shows an example of a breathing method presentation screen displayed in a breathing method presentation process according to the first embodiment.

FIG. 5 shows an example of a handling method presentation screen displayed in a handling method presentation process according to the first embodiment.

FIG. 6 is a block diagram showing the configuration of an information processing apparatus that forms the physical activity assistance system according to the first embodiment.

FIG. 7 is a flowchart showing a notification control process carried out by a physical activity assistance apparatus according to the first embodiment.

FIG. 8 is a block diagram showing the configuration of a control section of an information processing apparatus that forms a physical activity assistance system according to a second embodiment of the invention.

FIG. 9 is a diagrammatic view showing the configuration of a physical activity assistance system according to a third embodiment of the invention.

FIG. 10 shows an example of a warning screen displayed in a notification control process according to the third embodiment.

### DESCRIPTION OF EXEMPLARY EMBODIMENTS

### First embodiment

A first embodiment of the invention will be described below with reference to the drawings.

### Schematic Configuration of Physical Activity Assistance System

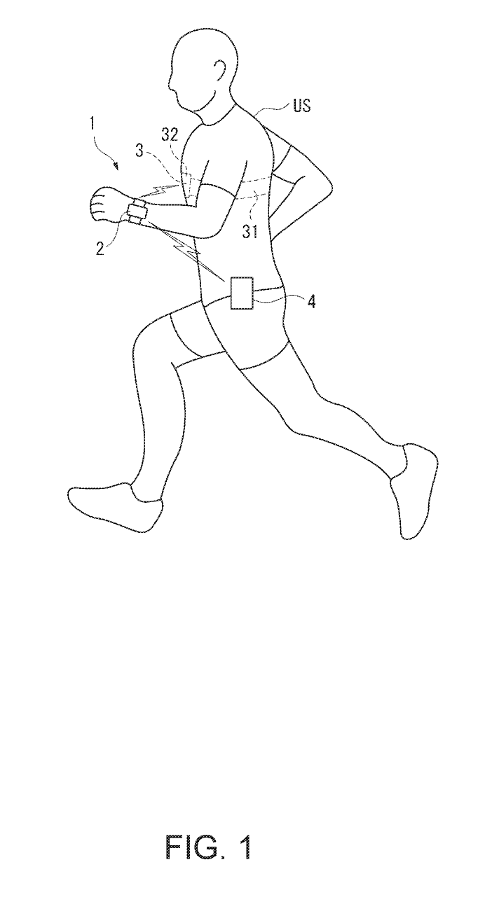

FIG. 1 is a diagrammatic view showing a schematic configuration of a physical activity assistance system 1 according to the present embodiment.

The physical activity assistance system 1 according to the present embodiment includes a biological information measurement apparatus 2 (hereinafter referred to as measurement apparatus in some cases), a breathing detection apparatus 3, which forms part of the measurement apparatus 2, and an information processing apparatus 4, which communicates with the measurement apparatus 2, as shown in FIG. 1. Among the components described above, the measurement apparatus 2 (instruction apparatus) and the breathing detection apparatus 3 correspond to the physical activity assistance apparatus according to an aspect of the invention.

The physical activity assistance system 1 is used by a user US who performs physical activity (mountain climbing, for example). In the physical activity assistance system 1, when the user US who is performing the physical activity is likely to be involved in an abnormal physical condition, the measurement apparatus 2 and the breathing detection apparatus 3 instruct the user to practice a predetermined breathing method, and even when the user US is still involved in the abnormal physical condition after the user US practices the breathing method, the measurement apparatus 2 and the breathing detection apparatus 3 instruct the user to practice a handling method for eliminating the abnormal physical condition.

Specifically, the physical activity assistance system 1 evaluates whether or not the user US is in danger of suffering from altitude sickness on the basis of biological information detected by the measurement apparatus 2 and the breathing detection apparatus 3, and when a result of the evaluation shows that the user US is in danger of suffering from altitude sickness, the physical activity assistance system 1 causes display sections of the measurement apparatus 2 and the breathing detection apparatus 3 to display a screen that instructs the user to practice the breathing method described above. Further, even after the user US practices the breathing method, when it is evaluated on the basis of the biological information described above, the breathing rate, and other factors that the user US is likely to be suffering from altitude sickness, the physical activity assistance system 1 causes the display sections of the measurement apparatus 2 and the breathing detection apparatus 3 to display a screen that instructs the user to descend the mountain.

The physical activity is not limited to mountain climbing and refers to general physical activity that involves motion of the body, such as high-altitude walking, running, and sports.

The measurement apparatus 2, the breathing detection apparatus 3, and the information processing apparatus 4 will be described below.

### Configuration of Biological Information Measurement Apparatus

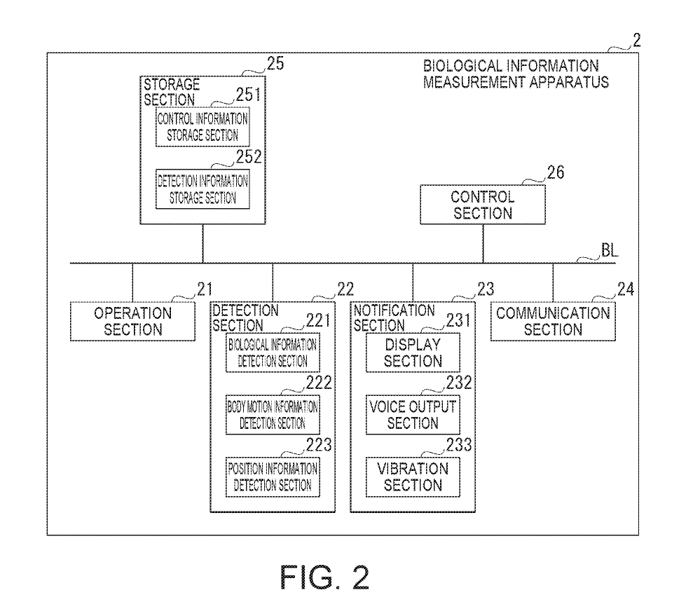

FIG. 2 is a block diagram showing the configuration of the biological information measurement apparatus 2.

The biological information measurement apparatus 2 is a wearable apparatus that is attached to the user US, as shown in FIG. 1, and detects and analyzes the user US's biological information and body motion information. The thus configured measurement apparatus 2 includes an operation section 21, a detection section 22, a notification section 23, a communication section 24, a storage section 25, and a control section 26, and these sections are connected to one another via a bus line BL, as shown in FIG. 2.

### Configuration of Operation Section

The operation section 21 has a plurality of buttons (not shown) disposed on an exterior case of the measurement apparatus 2 and outputs an operation signal according to a button that receives input operation (pressed-down button)to the control section 26. The operation section 21 does not necessarily have the configuration having buttons and may instead be a touch panel disposed on a display section 231, which will be described later, or may instead be configured to detect the user's tap operation.

### Configuration of Detection Section

The detection section 22 includes a biological information detection section 221, a body motion information detection section 222, and a position information detection section 223.

The biological information detection section 221 detects the user's biological information and outputs the biological information to the control section 26 under the control of the control section 26. The biological information detection section 221 detects the biological information in the form of the blood oxygen concentration and may be configured to further detect the pulse, heart rate, blood pressure, blood sugar level, body temperature, sweat rate, and other factors.

Specifically, the biological information detection section 221 applies infrared light to the blood flowing through the user US's blood vessel and detects the luminosity of light reflected off hemoglobin and oxyhemoglobin, which emit light in accordance with the applied infrared light.

The body motion information detection section 222 corresponds to the second sensor according to an aspect of the invention, detects body motion information representing the user's body motion, and outputs the body motion information to the control section 26 under the control of the control section 26. In the present embodiment, the body motion information detection section 222 detects the body motion information in the form of an acceleration signal that changes in accordance with the user's body motion. The body motion information detection section 222 may further detect angular velocity that changes in accordance with the user's body motion as well as the acceleration.

The position information detection section 223 acquires position information representing the current position of the measurement apparatus 2 (that is, position information representing current position of user US). For example, the position information detection section 223 corresponds to a satellite positioning system, such as a GPS (global positioning system), and acquires position information representing the current position described above on the basis of a radio wave received from a satellite. The position information detection section 223 then outputs the acquired position information to the control section 26. In place of the configuration described above, a configuration in which a communication radio wave is used to calculate the position information may be employed.

### Configuration of Notification Section

The notification section 23 notifies the user of a variety of pieces of information under the control of the control section 26. The notification section 23 includes a display section 231, a voice output section 232, and a vibration section 233.

The display section 231 has a display unit, such as a liquid crystal display panel or any of a variety of other display panels and a plurality of LEDs (light emitting diodes), and displays information inputted from the control section 26. For example, the display section 231 displays the biological information and the body motion information detected by the detection section 22 described above. The display section 231 further displays a warning screen (breathing method presenting screen and handling method presenting screen) and other screens based on presentation information generated by the control section 26.

The voice output section 232 includes a voice output unit, such as a loudspeaker, and outputs voice according to a voice signal inputted from the control section 26.

The vibration section 233 has a motor the action of which is controlled by the control section 26 and notifies the user of, for example, a warning in the form of vibration produced by the driven motor.

### Configuration of Communication Section

The communication section 24 includes a communication module that can communicate with the breathing detection apparatus 3 and the information processing apparatus 4 described above. The communication section 24 acquires data on the user US's breathing detected by the breathing detection apparatus 3. The communication section 24 transmits not only the biological information and the body motion information detected and acquired by the measurement apparatus 2 but also warning information that notifies the user US's abnormal physical condition and other pieces of information to the information processing apparatus 4.

In the present embodiment, the communication section 24 communicates with the information processing apparatus 4 in accordance with a short-range wireless communication scheme. Instead, the communication section 24 may communicate with the information processing apparatus 4 via a cradle or any other relay device or may communicate with the information processing apparatus 4 via a cable. The communication section 24 may still instead communicate with the information processing apparatus 4 and other external apparatus via a network.

### Configuration of Storage Section

The storage section 25 is formed of a storage unit including a flash memory or any other storage device and includes a control information storage section 251 and a detection information storage section 252.

The control information storage section 251 stores control information, such as a variety of programs and data necessary for the action of the measurement apparatus 2. As examples of the programs, the control information storage section 251 stores a control program that controls the measurement apparatus 2 and a notification control program for carrying out a physical activity assistance process including a breathing method notification process and a handling method notification process. The notification control process will be described later in detail.

The control information storage section 251 further stores, as the data described above, connection information necessary for communication connection with the breathing detection apparatus 3 and the information processing apparatus 4 and setting information inputted, for example, by the user. The setting information contains a first threshold and a second threshold of the blood oxygen concentration (SPO₂).

The first threshold and the second threshold are indices used to evaluate whether or not the user US is capable of mountain climbing. Out of the two thresholds, the first threshold is a value below which the user US is likely to suffer from altitude sickness and set and stored in advance by the user US or a medical worker. The second threshold is a value below which the user US has possibly suffered from altitude sickness and set and stored in advance by the user US or a medical worker, as in the case of the first threshold. For example, in the present embodiment, the first threshold is set at 100 SPO₂, and the second threshold is set at 80 SPO₂.

In the present embodiment, the first threshold and the second threshold can be changed as appropriate, as will be described later in detail, in accordance with the current location (altitude) where the user US is present.

The setting information described above further contains a breathing method practice limit period (10 minutes in initial state) and a restriction implementation limit period (3 minutes in initial state) in addition to the first threshold and the second threshold.

Among the parameters contained in the setting information, the breathing method practice limit period and the restriction implementation limit period are inputted and set by the user US or a medical worker via a setting screen displayed in the display section 23.

The detection information storage section 252 stores the biological information and the body motion information detected by the detection section 22 described above and results of analysis of the biological information and the body motion information (blood oxygen concentration and breathing rate, for example) performed by the control section 26. The detection information storage section 252 is configured to successively store these pieces of information and overwrite the information stored at the earliest point of time with newly acquired information in a case where the detection information storage section 252 is short of storage capacity.

### Configuration of Control Section

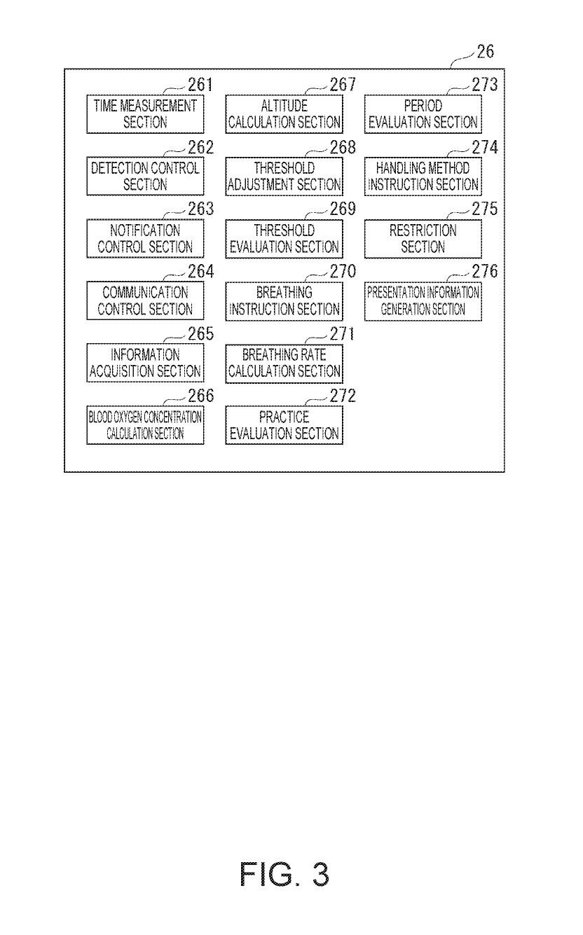

FIG. 3 is a block diagram showing the configuration of the control section 26.

The control section 26 includes a processing circuit, such as a CPU (central processing unit), and controls the action of the measurement apparatus 2 autonomously or in accordance with an operation signal inputted via the operation section 21 described above. The control section 26 has a time measurement section 261, a detection control section 262, a notification control section 263, a communication control section 264, an information acquisition section 265, a blood oxygen concentration calculation section 266, an altitude calculation section 267, a threshold adjustment section 268, a threshold evaluation section 269, a breathing instruction section 270, a breathing rate calculation section 271, a practice evaluation section 272, a period evaluation section 273, a handling method instruction section 274, a restriction section 275, and a presentation information generation section 276 as functional sections achieved when the processing circuit executes the program stored in the control information storage section 251, as shown in FIG. 3.

The time measurement section 261 measures the current date and time.

The detection control section 262 controls the action of the detection section 22 described above. For example, the detection control section 262, when it determines that the measurement apparatus 2 is not attached to the user, causes the biological information detection section 221 to stop detecting the biological information to lower power consumption.

The notification control section 263 controls the action of the notification section 23. For example, the notification control section 263 outputs notification information containing display and voice representing the state of the action of the measurement apparatus 2, results of the detection performed by the detection section 22, and other pieces of information to the notification section 23 to cause the notification section 23 to notify the notification information. The notification control section 263 further causes a setting screen where a variety of settings of the measurement apparatus 2 are inputted and changed to be displayed. The notification control section 263 further drives the motor of the vibration section 233 as required to notify predetermined information in the form of vibration produced by the driven motor.

The communication control section 264 controls the action of the communication section 24 described above.

The information acquisition section 265 acquires a variety of signals and a variety of pieces of information inputted from the operation section 21, the detection section 22, and the communication section 24. For example, the information acquisition section 265 acquires the content of an input to the setting screen described above on the basis of an operation signal according to input operation performed on the setting screen. The information acquisition section 265 further acquires the biological information (for example, information on luminosity of light reflected off hemoglobin and oxyhemoglobin in user US's blood), the body motion information, and the position information inputted from the detection section 22 and causes the storage section 25 (detection information storage section 252) to store these pieces of information. The information acquisition section 265 further acquires data on the breathing (breathing information) detected by the breathing detection apparatus 3 and causes the storage section 25 (detection information storage section 252) to store the data.

The blood oxygen concentration calculation section 266 calculates the user US's blood oxygen concentration on the basis of the biological information inputted from the biological information detection section 221. Specifically, the blood oxygen concentration calculation section 266 detects the user US's blood oxygen concentration on the basis of the information, detected by the biological information detection section 221 and acquired by the information acquisition section 265, on the luminosity of light reflected off hemoglobin and oxyhemoglobin in the blood flowing through the user US's blood vessel. The blood oxygen concentration of a typical user US who is at rest is, for example, 100 SPO₂.

The altitude calculation section 267 calculates the altitude at the current location where the user US is present on the basis of the position information detected by the position information detection section 223 and acquired by the information acquisition section 265.

The threshold adjustment section 268 has a function of adjusting the first threshold and the second threshold described above on the basis of the altitude calculated by the altitude calculation section 267. For example, when the altitude calculation section 267 shows that the altitude at the current location where the user US is present is lower than 1800 m above sea level, the threshold adjustment section 268 does not change (adjust) the first threshold (100 SPO₂) and the second threshold (80 SPO₂) set in advance. On the other hand, when the altitude at the current location where the user US is present is higher than or equal to 1800 m above sea level, the threshold adjustment section 268 sets the first threshold, for example, at 95 SPO₂ and adjusts the second threshold, for example, to 75 SPO₂.

It is noted that 100 SPO₂ means that a measured value of the blood oxygen concentration is 100%, and 80 SPO₂ means that a measured value of the blood oxygen concentration is 80%. Each of these expressions can instead be interpreted to mean that the expression represents a relative amount with respect to the blood oxygen concentration measured in a reference state. For example, assuming that the blood oxygen concentration measured at home is 100%, these expressions may be interpreted in such a way that 100 SPO₂ means that it is equal to the blood oxygen concentration measured at home, and 80 SPO₂ means that at corresponds to 80% the blood oxygen concentration measured at home.

The threshold evaluation section 269 has a function of evaluating whether or not the user US's blood oxygen concentration is higher than the first threshold and the second threshold. Specifically, the threshold evaluation section 269 evaluates whether or not the blood oxygen concentration detected by the blood oxygen concentration calculation section 266 is higher than the first threshold and the second threshold adjusted by the threshold adjustment section 268. In detail, the threshold evaluation section 269 performs the evaluation of whether or not the user US's blood oxygen concentration is higher than the first threshold, for example, every 5 minutes. Further, when the practice evaluation section 272, which will be described later, determines that the user US is practicing a pressure breathing method, the threshold evaluation section 269 evaluates whether or not the user US's blood oxygen concentration is higher than the first threshold. Further, when a result of the evaluation shows that the user US's blood oxygen concentration is not higher than the first threshold, the threshold evaluation section 269 evaluates whether or not the blood oxygen concentration is higher than the second threshold.

The breathing instruction section 270 has a function of instructing the user US to practice a pressure breathing method, which is a predetermined breathing method, when the threshold evaluation section 269 determines that the user US's blood oxygen concentration is not higher than the first threshold. Specifically, the breathing instruction section 270 causes the presentation information generation section 276, which will be described later, to generate a breathing method presentation screen.

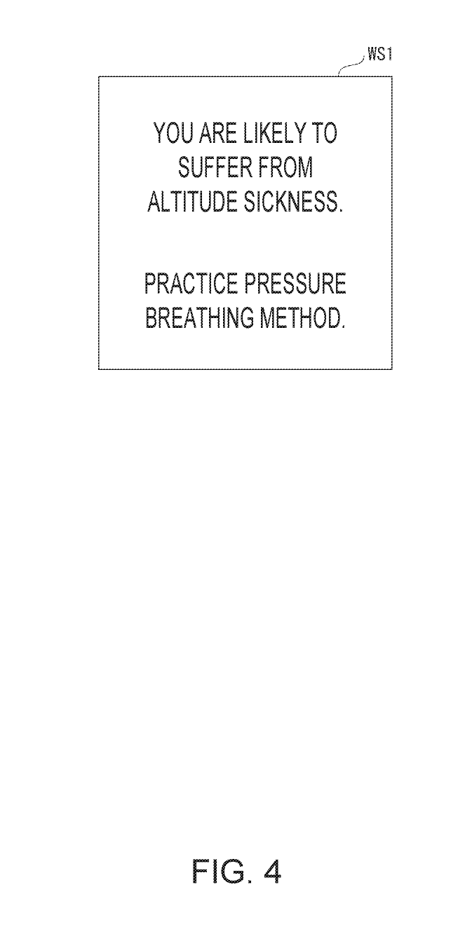

FIG. 4 shows a breathing method presentation screen WS1.

The breathing instruction section 270 causes the presentation information generation section 276 to generate the breathing method presentation screen WS1 shown in FIG. 4 and causes the display section 231 to display the breathing method presentation screen WS1. The breathing instruction section 270 further causes the communication control section 264 to transmit the breathing method presentation screen WS1 as warning information via the communication section 24 to the information processing apparatus 4. On the breathing method presentation screen WS1 are displayed letters that notify the user that the user is likely to suffer from altitude sickness and prompts the user to practice the pressure breathing method.

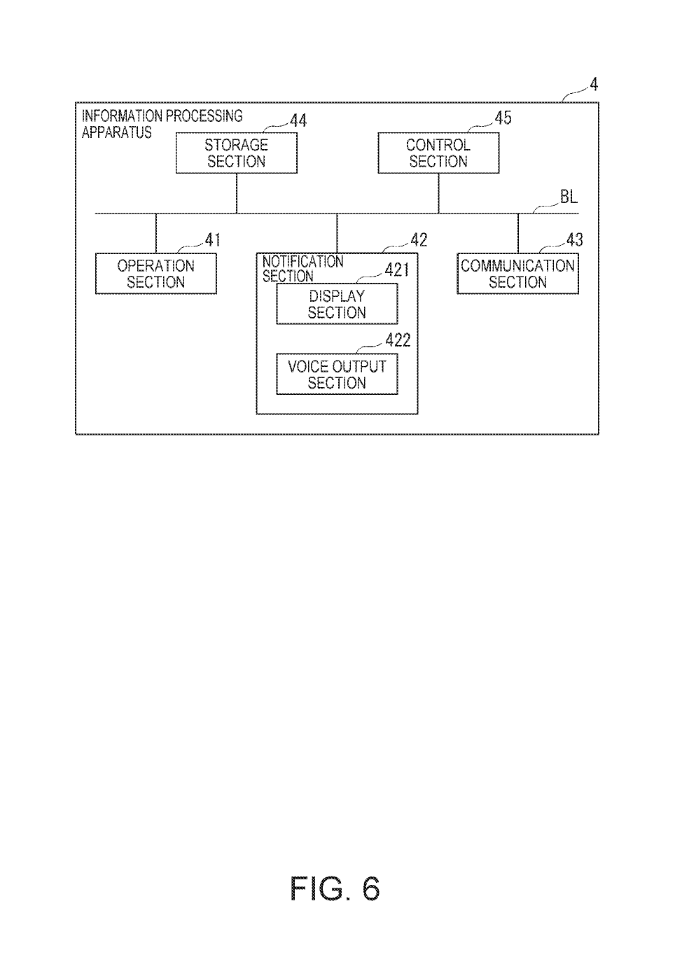

The pressure breathing method is a breathing method in which a person inhales a large amount of air, stops breathing for about 2 seconds, and exhales the inhaled air slowly out of the mouth while straining the abdomen and pressuring the chest so that the blood oxygen concentration is increased. Therefore, since the user US who visually recognizes the breathing method presentation screen WS1 displayed in the display section 231 and a display section 421 (see FIG. 6) of the information processing apparatus 4 is likely to practice the pressure breathing method, practicing the breathing method is likely to increase the user US's blood oxygen concentration.

The breathing instruction section 270 may instead transmit information representing that the user US needs to practice the pressure breathing method in place of the breathing method presentation screen WS1 as the warning information, and a control section 45 (see FIG. 6) of the information processing apparatus 4 may cause the breathing method presentation screen WS1 to be displayed on the basis of the warning information.

The breathing detection apparatus 3 will now be described in detail. The breathing detection apparatus 3 corresponds to the breathing detection section according to an aspect of the invention and detects breathing information on the breathing of the user US to whom the breathing detection apparatus 3 is attached. Specifically, the breathing detection apparatus 3 includes a band section 31 and a control section 32, as shown in FIG. 1. Out of the two sections, the band section 31, which is attached to the user US's chest, is configured to be expandable and contractible in accordance with the user US's breathing. The control section 32 acquires the breathing information on the user US's breathing in the form of data on the expansion/contraction of the band section 31 (data representing expansion/contraction in the form of wavelength, for example) and transmits the data to the measurement apparatus 2 via a communication section that is not shown. That is, the breathing detection apparatus 3 corresponds to the first sensor according to an aspect of the invention and detects the motion of the user US's chest.

Referring back to FIG. 3, the breathing rate calculation section 271 calculates the user US's breathing rate on the basis of the breathing information detected by the breathing detection apparatus 3 and stored in the storage section 25 and the body motion information. Specifically, the breathing rate calculation section 271 calculates the breathing rate over a predetermined period after the breathing instruction section 270 described above instructs the user to practice the pressure breathing method on the basis of the breathing information (motion of user US's chest) and the body motion information.

The practice evaluation section 272 evaluates whether or not the user US is practicing the pressure breathing method as instructed by the breathing instruction section 270. Specifically, the practice evaluation section 272 evaluates whether nor not the pressure breathing method has been practiced on the basis of the breathing rate over the predetermined period (3 minutes after the breathing instruction section 270 described above instructs the user to practice the pressure breathing method, for example) calculated by the breathing rate calculation section 271.

When the practice evaluation section 272 determines that the user US is practicing the pressure breathing method, the period evaluation section 273 acquires the period for which the user US has been practicing breathing according to the pressure breathing method and evaluates whether or not the period for which the user US has been practicing the pressure breathing method is longer than, for example, 10 minutes, that is, whether or not the breathing method practice limit period has elapsed. The period evaluation section 273 corresponds to the period acquisition section according to an aspect of the invention.

When the period evaluation section 273 determines that the acquired period is longer than the predetermined period, and the threshold evaluation section 269 determines that the user US's blood oxygen concentration is lower than or equal to the second threshold, the handling method instruction section 274 instructs the user to practice a handling method. Specifically, the handling method instruction section 274 causes the presentation information generation section 276, which will be described later, to generate a handling method presentation screen.

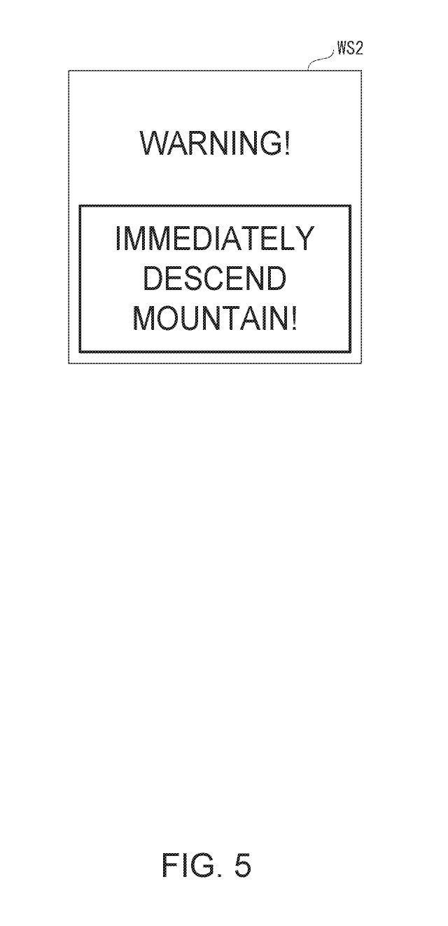

FIG. 5 shows a handling method presentation screen WS2.

The handling method instruction section 274 causes the presentation information generation section 276 to generate the handling method presentation screen WS2 shown in FIG. 5 and causes the communication control section 264 to transmit the handling method presentation screen WS2 to the information processing apparatus 4 via the communication section 24. On the handling method presentation screen WS2 are displayed letters that prompt the user to descend the mountain.

Therefore, the user US who visually recognizes the handling method presentation screen WS2 displayed in the display section 231 and the display section 421 of the information processing apparatus 4 is likely to descend the mountain, and descending the mountain is likely to increase the user US's blood oxygen concentration and improve the condition of the user who suffers from the altitude sickness.

The handling method instruction section 274 may instead transmit information representing that the user US is suffering from altitude sickness in place of the handling method presentation screen WS2 as the warning information, and the control section 45 of the information processing apparatus 4 may display the handling method presentation screen WS2 on the basis of the warning information.

Referring back to FIG. 3, when the user US's blood oxygen concentration is higher than the first threshold, the restriction section 275 restricts the instruction issued by the breathing instruction section 270. Specifically, the restriction section 275 restricts the display of the breathing method presentation screen WS1, which the breathing instruction section 270 has caused the presentation information generation section 276 to generate and which prompts the user to practice the pressure breathing method, in the information processing apparatus 4. For example, the restriction section 275 restricts the display of the breathing method presentation screen WS1 in the display section 231 and transmits a restriction signal that restricts the presentation of the breathing method presentation screen WS1 in the information processing apparatus 4 under the control of the communication control section 264.

The presentation information generation section 276 generates presentation information to be presented to the user. Specifically, the presentation information generation section 276 generates the breathing method presentation screen WS1 and the handling method presentation screen WS2 described above on the basis of instructions from the breathing instruction section 270 and the handling method instruction section 274.

### Configuration of Information Processing Apparatus

FIG. 6 is a block diagram showing the configuration of the information processing apparatus 4, which forms the physical activity assistance system according to the present embodiment.

The information processing apparatus 4 includes an operation section 41, a notification section 42, a communication section 43, a storage section 44, and the control section 45, and the sections 41 to 45 are electrically connected to one another via a bus line BL, as shown in FIG. 6.

### Configuration of Operation Section

The operation section 41 accepts an operator's input operation and outputs an operation signal according to the input operation to the control section 45, as in the operation section 21 described above does. The thus configured operation section 41 can be formed, for example, of physical keys or a touch panel provided on an enclosure of the information processing apparatus 4 or can be formed, for example, of a keyboard and a pointing device connected to the information processing apparatus 4 in a wired or wireless manner.

### Configuration of Notification Section

The notification section 42 notifies the operator of a variety of pieces of information under the control of the control section 45. The notification section 42 includes the display section 421 and a voice output section 422.

The display section 421 has a liquid crystal display panel or any of a variety of other display panels and displays a predetermined image under the control of a notification control section 452, which will be described later. Examples of the image may include an image generated by a presentation information generation section 465, which will be described later.

The voice output section 42 includes a voice output unit, such as a loudspeaker, and outputs voice according to voice information inputted from the control section 45.

The notification section 42 includes no vibration section, unlike the notification section 23 of the measurement apparatus 2 described above. However, when the information processing apparatus 4 is formed, for example, of a smartphone or a tablet, the notification section 42 may include a vibration section.

### Configuration of Communication Section

The communication section 43 includes a first communication module that can communicate with the measurement apparatus 2 described above and a second communication module that can communicate with an external apparatus on a network, such as the Internet, and communicates with both the apparatus under the control of the control section 45. The communication section 43 only needs to include one of the first communication module and the second communication module in a case where the communication section 43 can communicate with the external apparatus in the same communication scheme in accordance with which the communication section 43 communicates with the measurement apparatus 2. Further, in a case where the communication section 43 does not need to communicate with an external apparatus on a network, the communication section 43 does not need to include the second communication module.

### Configuration of Storage Section

The storage section 44 is formed, for example, of an HDD (hard disk drive), an SSD (solid state drive), or a flash memory and stores a variety of programs and data necessary for the action of the information processing apparatus 4. As an example of the programs, the storage section 44 stores a control program that controls the information processing apparatus 4. The storage section 44 further stores, as the setting information described above, for example, connection information for communication connection with the measurement apparatus 2 described above.

### Configuration of Control Section

The control section 45 includes a CPU (central processing unit) and executes the program stored in the storage section 44 to control the action of the information processing apparatus 4. For example, when the measurement apparatus 2 transmits the breathing method presentation screen WS1 and the handling method presentation screen WS2 as a warning signal, and the control section 45 receives the presentation screens WS1 and WS2, the control section 45 causes the display section 421 to display the breathing method presentation screen WS1 and the handling method presentation screen WS2.

The thus configured information processing apparatus 4 is connected to the measurement apparatus 2 via the communication section 43, and the measurement apparatus 2 along with the information processing apparatus 4 carries out a notification control process shown below.

### Notification Control Process

The control section 26 of the measurement apparatus 2 described above executes the notification control program stored in the control information storage section 251 to carry out a breathing method presentation process and a handling method presentation process contained in the notification control process (physical activity assistance process) independently of each other. That is, the notification control process is a process including the physical activity assistance method according to an aspect of the invention.

The notification control process will be described below in detail.

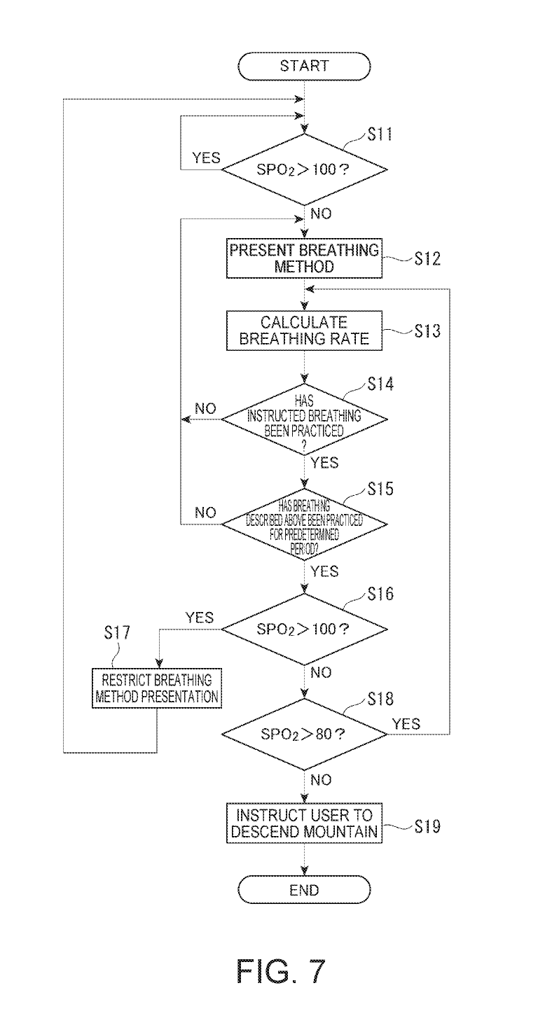

FIG. 7 is a flowchart showing the notification control process.

The notification control process is a physical activity assistance process including the breathing method presentation process for presenting the pressure breathing method and the handling method presentation process for presenting a handling method in accordance with the user US's blood oxygen concentration.

As preprocessing performed before the notification control process described above is carried out, the threshold adjustment section 268 adjusts the first threshold and the second threshold on the basis of the altitude calculated by the altitude calculation section 267 described above. For example, the first threshold is adjusted to 100 SPO₂, and the second threshold is set at 80 SPO₂.

When the user US starts physical activity, the threshold evaluation section 269 performs the evaluation of whether or not the user US's blood oxygen concentration is higher than the first threshold (100 SPO₂), for example, every 5 minutes (step S11), as shown in FIG. 7. When a result of the evaluation in the evaluation process instep S11 shows that the user US's blood oxygen concentration is higher than the first threshold, the threshold evaluation section 269 continues carrying out the process in the step S11 every 5 minutes.

On the other hand, when a result of the evaluation in the evaluation process in step S11 shows that the user US's blood oxygen concentration is not higher than the first threshold, the breathing instruction section 270 causes the presentation information generation section 276 to generate the breathing method presentation screen WS1, causes the display section 231 to display the breathing method presentation screen WS1, and transmits the breathing method presentation screen WS1 via the communication section 24 to the information processing apparatus 4 to cause the display section 421 of the information processing apparatus 4 to display the breathing method presentation screen WS1 (step S12).

When the process in step S12 is carried out, the breathing rate calculation section 271 calculates the breathing rate over the predetermined period after the breathing instruction section 270 instructs the user to practice the pressure breathing method on the basis of the breathing information and the body motion information described above (step S13).

The practice evaluation section 272 then evaluates whether or not the pressure breathing method has been practiced on the basis of the breathing rate over the predetermined period calculated by the breathing rate calculation section 271 (step S14). When a result of the evaluation in step S14 shows that the pressure breathing method has not been practiced, the breathing instruction section 270 carries out the processes in steps S12 and S13 again.

On the other hand, when a result of the evaluation in step S14 shows that the pressure breathing method has been practiced, the practice evaluation section 272 acquires the period for which the user US has been practicing breathing according to the pressure breathing method and evaluates whether or not the period for which the user US has been practicing the pressure breathing method is longer than the predetermined period (step S15). When a result of the evaluation instep S15 shows that the period for which the user US has been practicing the pressure breathing method is not longer than the predetermined period, the breathing instruction section 270 carries out the process in step S12 again.

On the other hand, when a result of the evaluation in step S15 shows that the period for which the user US has been practicing the pressure breathing method is longer than the predetermined period, the threshold evaluation section 269 evaluates whether or not the user US's blood oxygen concentration is higher than the first threshold (100 SPO₂) (step S16).

When a result of the evaluation in step S16 shows that the user US's blood oxygen concentration is higher than the first threshold, the restriction section 275 restricts the display of the breathing method presentation screen WS1, which the breathing instruction section 270 has caused the presentation information generation section 276 to generate and which prompts the user to practice the pressure breathing method, in the display section 231 and the display section 421 of the information processing apparatus 4 (step S17). The control then returns to the process in step S11.

On the other hand, when a result of the evaluation in step S16 shows that the user US's blood oxygen concentration is not higher than the first threshold, the threshold evaluation section 269 evaluates whether or not the user US's blood oxygen concentration is higher than the second threshold (80 SPO₂) (step S18).

When a result of the evaluation in step S18 shows that the user US's blood oxygen concentration is higher than the second threshold, the control returns to the process in step S13, where the process described above is carried out.

On the other hand, when a result of the evaluation in step S18 shows that the user US's blood oxygen concentration is not higher than the second threshold, the handling method instruction section 274 causes the presentation information generation section 276 to generate the handling method presentation screen WS2, causes the display section 231 to display the handling method presentation screen WS2, and causes the communication control section 264 to transmit the handling method presentation screen WS2 to the information processing apparatus 4 via the communication section 24 to cause the display section 421 of the information processing apparatus 4 to display the handling method presentation screen WS2 (step S19).

After the handling method presentation process in step S19 is carried out, the notification control process is completed.

### Advantageous Effects of First Embodiment

The physical activity assistance system 1 according to the present embodiment described above provides the following advantageous effects.

Since the first threshold is set at a value below which the user is likely to suffer from altitude sickness (100 SPO₂, for example), the user US is instructed, when the user US's blood oxygen concentration is lower than or equal to the first threshold, to practice the pressure breathing method, which allows the blood oxygen concentration to rise, whereby the user can lower a risk of developing altitude sickness by practicing breathing in accordance with the instruction. Therefore, when the user US practices the breathing method to successfully cause the blood oxygen concentration to be higher than the first threshold, the user US can continue the physical activity. The physical activity assistance system 1 can therefore appropriately support the user US's physical activity.

In a case where the blood oxygen concentration is lower than or equal to the second threshold (80 SPO₂), which is lower than the first threshold (100 SPO₂), even after the user US practices the pressure breathing method, the user is instructed to descend the mountain as the handling method, whereby a situation in which the user US continues the mountain climbing and is involved in a risky state (altitude sickness, for example) can be avoided. The physical activity assistance system 1 can therefore appropriately support the user US's physical activity (mountain climbing).

In a case where the blood oxygen concentration is not higher than the second threshold even after the user US practices the pressure breathing method for the predetermined period, the user is instructed to descend the mountain as the handling method, whereby a situation in which the user US continues the mountain climbing and is involved in a risky state can be further avoided. The physical activity assistance system 1 can therefore more appropriately support the user US's physical activity (mountain climbing).

Since the breathing detection apparatus 3 as the first sensor can detect the motion of the user US's chest, the user US's breathing rate can be readily measured. The physical activity assistance system 1 can therefore evaluate on the basis of the breathing rate whether or not the user US is appropriately practicing the pressure breathing method. The physical activity assistance system 1 can therefore more adequately provide the user US with a handling method instruction.

Since the physical activity assistance system 1 evaluates whether or not the user US is practicing the pressure breathing method on the basis of the motion of the user US's body motion information and chest motion (breathing information), the physical activity assistance system 1 can more accurately evaluate whether or not the user is practicing the pressure breathing method. The physical activity assistance system 1 can therefore further adequately provide the user US with a handling method instruction.

Since the threshold adjustment section 268 can adjust the first threshold and the second threshold on an altitude basis, the physical activity assistance system 1 can adequately provide the user US with breathing method and handling method instructions in accordance with the altitude.

Since the instruction issued by the breathing instruction section 270 is restricted when the blood oxygen concentration is higher than the first threshold, that is, when the user US's blood oxygen concentration reaches an appropriate value, the user US can recognize that the blood oxygen concentration has reached the appropriate value. The fact that the display section 231 and the display section 421 stop displaying the instruction therefore allows the user US to readily recognize that the user US can continue the physical activity (mountain climbing, for example).

### Second Embodiment

A second embodiment of the invention will next be described.

A biological information measurement apparatus that forms a physical activity assistance system according to the present embodiment has a configuration similar to that of the biological information measurement apparatus 2 described above, but part of the control section 26 is configured differently. Further, an information processing apparatus that form the physical activity assistance system according to the present embodiment has a configuration similar to that of the information processing apparatus 4 described above, but part of the control section is configured differently. Specifically, the information processing apparatus according to the present embodiment differs from the information processing apparatus 4 in that it acquires the biological information, the body motion information, and the position information detected by the measurement apparatus and uses these pieces of information to carry out the same notification control process (physical activity assistance process) carried out by the biological information measurement apparatus 2. The biological information measurement apparatus and the information processing apparatus according to the present embodiment differ from the biological information measurement apparatus 2 and the information processing apparatus 4 described above with regard to these points. In the following description, the same or roughly the same portions as those having already been described have the same reference characters and will not be described.

### Configuration of Biological Information Measurement Apparatus

The biological information measurement apparatus according to the present embodiment has a configuration similar to that of the biological information measurement apparatus 2 described above. The control section of the biological information measurement apparatus according to the present embodiment does not include the blood oxygen concentration calculation section 266, the altitude calculation section 267, the threshold adjustment section 268, the threshold evaluation section 269, the breathing instruction section 270, the breathing rate calculation section 271, the practice evaluation section 272, the period evaluation section 273, the handling method instruction section 274, the restriction section 275, or the presentation information generation section 276 described above.

### Configuration of Information Processing Apparatus

An information processing apparatus 4A according to the present embodiment has a configuration similar to that of the information processing apparatus 4 described above. Specifically, the information processing apparatus 4A includes the operation section 41, the notification section 42, the communication section 43, and the storage section 44 and further includes a control section 450 in place of the control section 45 described above.

### Configuration of Control Section

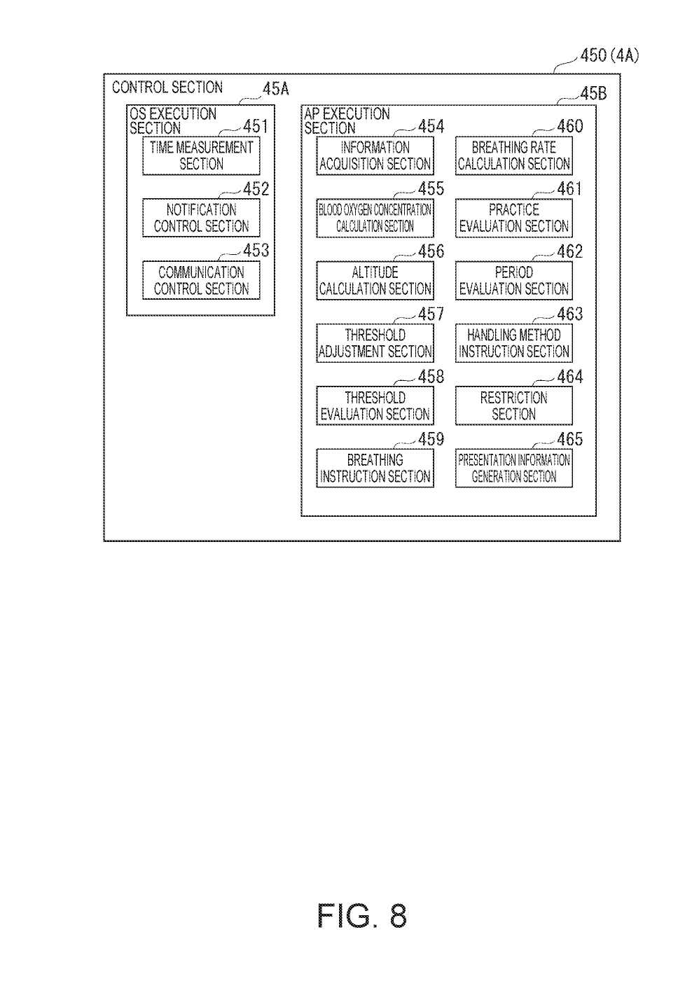

FIG. 8 is a block diagram showing the configuration of the control section 450.

The control section 450 includes a CPU (central processing unit) and executes a program stored in the storage section 44 to control the action of the information processing apparatus 4A. The thus configured control section 450 includes an OS execution section 45A and an AP execution section 45B, as shown in FIG. 8.

### Configuration of OS Execution Section

The OS execution section 45A is a functional section that executes an OS stored in the storage section 44 and includes a time measurement section 451, a notification control section 452, and a communication control section 453.

The time measurement section 451 measures the current date and time.

The notification control section 452 includes, although not shown, a display control section and a voice output control section. Out of the two sections, the display control section acquires, from the AP execution section 45B, an image of a screen displayed when a program, such as the OS or any of a variety of applications, is executed and causes the display section 421 to display the image. The voice output control section outputs a voice signal generated when the program is executed to cause the voice output section 422 to output voice according to the voice signal.

The communication control section 453 controls communication between the communication section 43 and an external apparatus.

### Configuration of AP Execution Section

The AP execution section 45B executes, among applications stored in the storage section 44, an application specified by the OS execution section 45A in accordance with an operation signal inputted via the operation section 41. The AP execution section 45B includes an information acquisition section 454, a blood oxygen concentration calculation section 455, an altitude calculation section 456, a threshold adjustment section 457, a threshold evaluation section 458, a breathing instruction section 459, a breathing rate calculation section 460, a practice evaluation section 461, a period evaluation section 462, a handling method instruction section 463, a restriction section 464, and a presentation information generation section 465, each of which functions when the CPU executes the physical activity assistance application that causes the AP execution section 45B to carry out the notification control process.

The information acquisition section 454 acquires a variety of signals and a variety of pieces of information inputted through the operation section 41 and the communication section 43. For example, the information acquisition section 454 acquires the content of an input to the setting screen described above on the basis of an operation signal according to input operation performed on the setting screen. The information acquisition section 454 further acquires the biological information, the body motion information, and the position information received from the measurement apparatus 2 and causes the storage section 44 to store these pieces of information. The information acquisition section 454 further acquires data on the breathing detected by the breathing detection apparatus 3 (breathing information) and causes the storage section 44 to store the data.

The blood oxygen concentration calculation section 455 calculates the user US's blood oxygen concentration on the basis of the biological information acquired by the information acquisition section 454.

The altitude calculation section 456 calculates the altitude at the current location where the user US is present on the basis of the position information acquired by the information acquisition section 454.

The threshold adjustment section 457 adjusts the first threshold and the second threshold described above on the basis of the altitude calculated by the altitude calculation section 456.

The threshold evaluation section 458 evaluates whether or not the user US's blood oxygen concentration is higher than the first threshold and the second threshold.

The breathing instruction section 459 instructs the user US to practice the pressure breathing method, which is the predetermined breathing method, when the threshold evaluation section 458 determines that the user US's blood oxygen concentration is lower than or equal to the first threshold.

The breathing rate calculation section 460 calculates the user US's breathing rate on the basis of the breathing information and the body motion information detected by the breathing detection apparatus 3 and stored in the storage section 25.

The practice evaluation section 461 evaluates whether or not the user US is practicing the pressure breathing method that the user has been instructed to practice by the breathing instruction section 459.

When the practice evaluation section 461 determines that the user US is practicing the pressure breathing method, the period evaluation section 462 acquires the period for which the user US has been practicing breathing according to the pressure breathing method and evaluates whether or not the period for which the user US has been practicing the pressure breathing method is longer than, for example, 10 minutes.

When the period evaluation section 462 determines that the acquired period is longer than the predetermined period, and the threshold evaluation section 458 determines that the user US's blood oxygen concentration is lower than or equal to the second threshold, the handling method instruction section 463 instructs the user to practice a handling method.

When the user US's blood oxygen concentration is higher than the first threshold, the restriction section 464 restricts the instruction issued by the breathing instruction section 459.

The presentation information generation section 465 generates presentation information to be presented to the user.

The control section 450 described above executes a notification control program stored in the storage section 44 to carry out the breathing method presentation process and the handling method presentation process contained in the notification control process (physical activity assistance process) independently of each other. That is, the control section 450 carries out the processes described above in accordance with the flowchart shown in FIG. 7 to assist the user US's physical activity, as the control section 26 of the measurement apparatus 2 in the first embodiment does.

### Advantageous Effects of Second Embodiment

The physical activity assistance system according to the second embodiment described above provides the following advantageous effects as well as the same advantageous effects provided by the physical activity assistance system I described above.

In the present embodiment, the information processing apparatus 4A carries out the physical activity assistance process (notification control process) including the breathing method presentation process and the handling method presentation process described above, which are processes that consume a large amount of electric power and impose a heavy processing burden on the CPU, whereby the biological information measurement apparatus according to the present embodiment can be operated for a relatively long period, and cost reduction is achieved.

### Third Embodiment

A third embodiment of the invention will next be described.

A physical activity assistance system according to the present embodiment has a configuration similar to that of the physical activity assistance system according to the second embodiment described above but differs therefrom in that it includes a plurality of biological information measurement apparatus and information processing apparatus that are the same as the biological information measurement apparatus and the information processing apparatus that form the physical activity assistance system according to the second embodiment and that the plurality of information processing apparatus are so connected to one another as to communicate with one another. In the following description, the same or roughly the same portions as those having already been described have the same reference characters and will not be described.

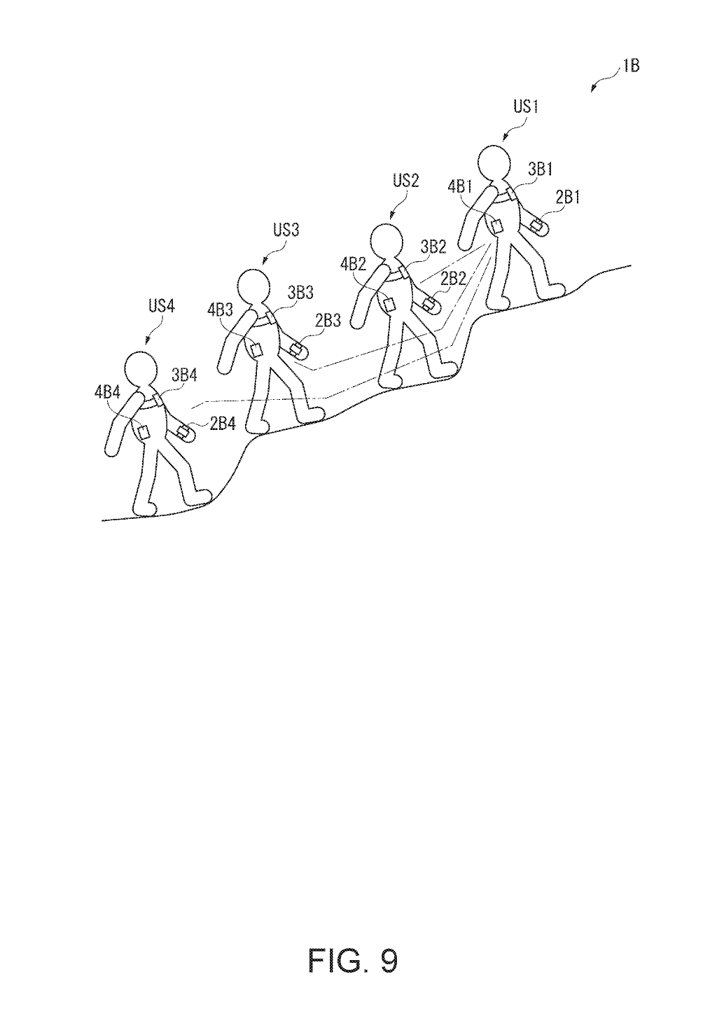

FIG. 9 is a diagrammatic view showing a physical activity assistance system 1B according to the present embodiment.

The physical activity assistance system 1B according to the present embodiment includes a biological information measurement apparatus 2B1 (hereinafter referred to as measurement apparatus in some cases), a breathing detection apparatus 3B1, and an information processing apparatus 4B1, which are used by a user US1, a measurement apparatus 2B2, a breathing detection apparatus 3B2, and an information processing apparatus 4B2, which are used by a user US2, a measurement apparatus 2B3, a breathing detection apparatus 3B3, and an information processing apparatus 4B3, which are used by a user US3, and a measurement apparatus 2B4, a breathing detection apparatus 3B4, and an information processing apparatus 4B4, which are used by a user US4, as shown in FIG. 9.

Each of the plurality of measurement apparatus 2B1 to 2B4 has the same configuration as that of the measurement apparatus according to the second embodiment. Each of the plurality of breathing detection apparatus 3B1 to 3B4 has the same configuration as that of the breathing detection apparatus 3 described above. Each of the plurality of information processing apparatus 4B1 to 4B4 has the same configuration as that of the information processing apparatus 4A described above. In addition, the information processing apparatus 4B1 among the plurality of information processing apparatus 4B1 to 4B4 is connected to the other information processing apparatus 4B2 to 4B4 via the communication sections 43.

For example, when a foursome party (unit) formed of the users US1 to US4 climbs a mountain, a leader walks first and a subleader walks last in many cases. In this case, the user US1, who is the leader, needs to grasp the body conditions of the other users US2 to US4, who form the party. To this end, for example, when the users US1 to US4 form one party and climb a mountain, the information processing apparatus 4B1 to 4B4 are so set before the mountain climbing that the information processing apparatus 4B1 of the user US1, who is the leader, receives measurement information and warning information transmitted from the other information processing apparatus 4B2 to 4B4.

Specifically, when the control section 450 of any of the information processing apparatus 4B2 to 4B4 causes the breathing method presentation screen WS1 to be displayed in the display section 421 of the corresponding one of the information processing apparatus 4B2 to 4B4, an instruction issued by the breathing instruction section 459 is transmitted as the warning information to the information processing apparatus 4B1 of the user US1 via the communication section 43. The control section 450 of the information processing apparatus 4B1, when it acquires the warning information, causes the presentation information generation section 465 to generate a warning screen WS3 and causes the display section 421 of the information processing apparatus 4B1 to display the warning screen WS3.

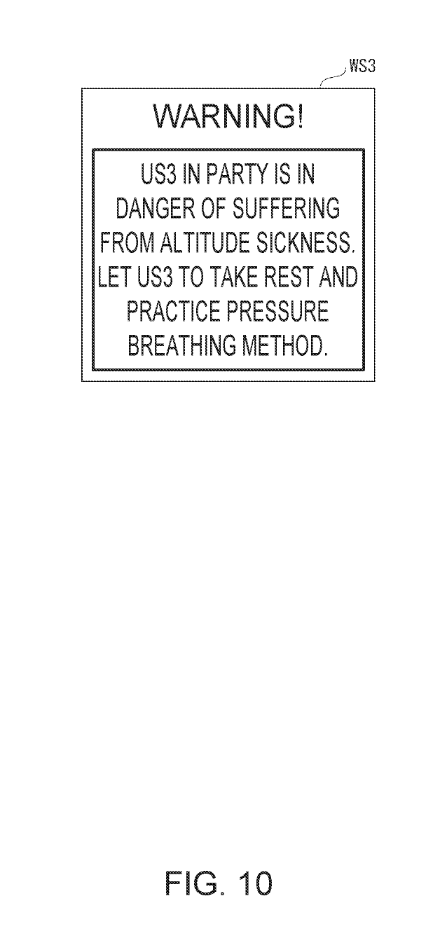

FIG. 10 shows the warning screen WS3.

The control section 450 of the information processing apparatus 4B1 causes the presentation information generation section 465 to generate the warning screen WS3 shown in FIG. 10 and causes the display section 421 to display the warning screen WS3. On the warning screen WS3 are displayed letters that state that the user US3, who carries the information processing apparatus 4B3, which has transmitted the warning information, is likely to suffer from altitude sickness and prompt the user US3 to practice the pressure breathing method. Therefore, since the user US1 who visually recognizes the warning screen WS3 is likely to prompt the user US3 to practice the pressure breathing method, practicing the breathing method is likely to increase the user US3′s blood oxygen concentration.

Similarly, when the control section 450 of any of the information processing apparatus 4B2 to 4B4 causes the handling method presentation screen WS2 to be displayed in the display section 421 of the corresponding one of the information processing apparatus 4B2 to 4B4, an instruction issued by the handling method instruction section 463 is transmitted as the warning information to the information processing apparatus 4B1 of the user US1 via the communication section 43 as in the case in which the breathing method presentation screen WSI is displayed. The control section 450 of the information processing apparatus 4B1, when it acquires the warning information, causes the presentation information generation section 465 to generate a warning screen (not shown) and causes the display section 421 of the information processing apparatus 4B1 to display the warning screen.

For example, the control section 450 causes the display section 421 to display a warning screen on which information stating that the user U53 has suffered from altitude sickness and is instructed to descend the mountain is displayed.

### Advantageous Effects of Third Embodiment

The physical activity assistance system 1B according to the third embodiment described above provides the following advantageous effects as well as the same advantageous effects provided by the physical activity assistance system 1 described above.

When the information processing apparatus 4B1 of the user US1, who is the leader of the party, receives the warning information described above transmitted from any of the information processing apparatus 4B2 to 4B4 of the other users US2 to US4, the warning screen WS3 is displayed in the display section 421. The user US1 can therefore grasp both a state in which one of the members (users US2 to US4) of the party is likely to suffer from altitude sickness and a state in which the member has suffered from altitude sickness.

Further, since the user US1 can readily visually recognize a handling method to be taken in the state in which any of the users US2 to US4 is likely to suffer from altitude sickness and the state in which the user has suffered from altitude sickness, a situation in which any of the other users US2 to US4 is involved in a risky state can be avoided. The user US1 can therefore assist and help the physical activities of the users US2 to US4.

### Variations of Embodiments

The invention is not limited to the embodiments described above, and variations, improvements, and other modifications to the extent that the advantage of the invention is achieved fall within the scope of the invention.

In each of the embodiments described above, the breathing detection apparatus 3 is provided. The invention does not, however, necessarily have the configuration. For example, the breathing detection apparatus 3 may not be provided. In this case, the control section 26 or 45 may not include the breathing rate calculation section 271 or 460 or the practice evaluation section 272 or 461, and after a predetermined period elapses since the breathing instruction section 459 displayed the breathing method presentation screen WS1 in the display section 231 and the display section 421 of the information processing apparatus 4 or 4A, and the evaluation of whether or not the user US's blood oxygen concentration is lower than or equal to the second threshold performed by the threshold evaluation section 269 shows that the blood oxygen concentration is lower than or equal to the second threshold, the handling method instruction sections 274 and 463 may display the handling method presentation screen WS2 in the display section 231 and the information processing apparatus 4. That is, in place of the processes in step S13 to S15 in the flowchart of FIG. 7, the process of evaluating whether or not the predetermined period has elapsed since the breathing method presentation screen WS1 was displayed may be carried out, and the processes in step S16 and the following steps may be carried out in a case where the predetermined period has elapsed in the process.

In each of the embodiments described above, the control sections 26 and 450 include the handling method instruction sections 274 and 463, respectively. The invention does not, however, necessarily have the configuration. For example, the control section 26 or 450 may not include the handling method instruction section 274 or 463. Even in this case, since the breathing instruction sections 270 and 459 cause the breathing method presentation screen WS1 to be displayed in the display section 231 and the display section 421 of the information processing apparatus 4 or 4A, the user US' s physical activity can be assisted. That is, the processes in steps S18 and S19 in the flowchart shown in FIG. 7 may not be carried out.

In each of the embodiments described above, the control sections 26 and 450 include the period evaluation sections 273 and 462, respectively, each of which acquires, when it is determined that the user US is practicing breathing according to the pressure breathing method, the period for which the user US has been practicing the breathing according to the pressure breathing method and evaluates whether or not the period is longer than a predetermined period. The invention does not, however, necessarily have the configuration. For example, the control section 26 or 450 may not include the period evaluation section 273 or 462. In this case, the handling method instruction sections 274 and 463 may instruct the user US to practice a handling method when it is determined that the user US is practicing the breathing according to the pressure breathing method and the measured blood oxygen concentration is lower than or equal to the second threshold. That is, the process in step S15 in the flowchart shown in FIG. 7 may not be carried out. Further, the breathing method is not limited to the pressure breathing method and may be deep breathing, abdominal breathing, and other types of breathing that allow the user to inhale oxygen through the breathing.

In each of the embodiments described above, the breathing rate calculation sections 271 and 460 calculate the breathing rate on the basis of the breathing information detected by the breathing detection apparatus 3 and the body motion information detected by the body motion information detection section 222. The invention does not, however, necessarily have the configuration. For example, the breathing rate calculation sections 271 and 460 may calculate the breathing rate on the basis only of the breathing information. That is, the measurement apparatus 2 may not include the body motion information detection section 222.

In each of the embodiments described above, the breathing detection apparatus 3 includes the band section 31 and the control section 32, and the breathing rate calculation sections 271 and 460 calculate the breathing rate from the wavelength (breathing information) based on the extraction/contraction of the band section 31. The invention does not, however, necessarily have the configuration. For example, instead of the breathing detection apparatus 3, the biological information detection section 221 may detect an electrocardiogram and perform frequency analysis on the electrocardiogram to calculate the breathing rate. The biological information detection section 221 may still instead acquire a breathing component from the waveform of the electrocardiogram and analyze the breathing component to calculate the breathing rate.

In each of the embodiments described above, when the altitude of the current location where the user US is present is higher than or equal to 1800 m, the first threshold and the second threshold are adjusted. The invention does not, however, necessarily have the configuration. For example, when the altitude described above is higher than or equal to 2500 m, the first threshold and the second threshold may be adjusted, or the first threshold and the second threshold may be lowered as the altitude increases.

Instead, the first threshold and the second threshold may not be adjusted. In this case, the control section 26 may not include the altitude calculation section 267 or 456 or the threshold adjustment section 268 or 457. Further, for example, the processes carried out by the altitude calculation sections 267 and 456 may be carried out every one hour, and when the altitude calculated by the altitude calculation sections 267 and 456 changes from the altitude acquired in the preceding calculation by at least a predetermined value, the process by the threshold adjustment section 268 may be carried out.

In each of the embodiments described above, the threshold adjustment sections 268 and 457 adjust the first threshold and the second threshold on the basis of the current location (altitude) where the user US is present and which is calculated by the altitude calculation sections 267 and 456. The invention does not, however, necessarily have the configuration. For example, the first threshold and the second threshold may be set in advance in accordance with the user US's physical strength or may be changed in accordance with the user US's physical strength and the altitude. In this case, the measurement apparatus 2 may not include the position information detection section 223 or the altitude calculation section 267 or 456.

The threshold adjustment sections 268 and 457 may set the user US's blood oxygen concentration before mounting climbing to be the first threshold and may set the second threshold at a value corresponding to the first threshold. Further, the first threshold and the second threshold may be so set as not to be changed from values set in advance or may be inputted and set by the user US and a medical worker via the setting screen displayed in the display section 231.

Further, the threshold adjustment sections 268 and 457 adjust both the first threshold and the second threshold in the above description. The invention does not, however, necessarily have the configuration. For example, the threshold adjustment sections 268 and 457 may adjust only one of the first threshold and the second threshold.

In each of the embodiments described above, on the breathing method presentation screen WS1 are displayed only letters that prompt the user to practice the pressure breathing method. The invention does not, however, necessarily have the configuration. For example, in place of or in addition to the letters described above, a pattern, video images, and other objects that prompt the user to practice the pressure breathing method may be displayed. Instead, an explanatory note for allowing the user to practice the pressure breathing method and a pattern, video images, and other objects that explain the pressure breathing method may be displayed.

In each of the embodiments described above, the control sections 26 and 450 include the restriction sections 275 and 464, respectively. The invention does not, however, necessarily have the configuration. For example, the control section 26 or 450 may not include the restriction section 275 or 464.

In each of the embodiments described above, the measurement apparatus 2 includes the biological information detection section 221, the body motion information detection section 222, and the position information detection section 223 as the detection section 22. The invention does not, however, necessarily have the configuration. For example, the measurement apparatus 2 may additionally include an atmospheric pressure detection section that detects the atmospheric pressure. The atmospheric pressure detection section allows the altitude calculation section 267 to calculate the altitude on the basis of the position information and the atmospheric pressure information. Further, the handling method instruction section 274 instructs the user US to descend the mountain when the user US's blood oxygen concentration is lower than the second threshold. The invention does not, however, necessarily have the configuration. For example, the handling method instruction section 274 may instruct the user to descend the mountain when it is likely to rain on the basis of the atmospheric pressure information or when it is likely to rain even in a case where the user US's blood oxygen concentration is higher than or equal to the second threshold but lower than the first threshold.

In each of the embodiments described above, the breathing method presentation screen WS1 and the handling method presentation screen WS2 are displayed in the display section 241 and the display section 421 of the information processing apparatus 4 or 4A. The invention does not, however, necessarily have the configuration. For example, the breathing method presentation screen WS1 and the handling method presentation screen WS2 may be displayed only in the display section 241 of the measurement apparatus 2 or the display section 421 of the information processing apparatus 4 or 4A. For example, in the case where the breathing method presentation screen WS1 and the handling method presentation screen WS2 are displayed only in the display section 241, the physical activity assistance system 1 described above is formed only of the measurement apparatus 2.

Further, when the breathing method presentation screen WS1 and the handling method presentation screen WS2 are displayed in the display section 231 of the measurement apparatus 2, the voice output section 232 may be allowed to output voice, or the vibration section 233 may be allowed to vibrate.

In each of the embodiments described above, mountain climbing is exemplified as the physical activity. The invention does not, however, relate to mountain climbing. For example, in a case where the physical activity is any other physical activity, when the blood oxygen concentration is higher than the first threshold, a breathing method suitable for that physical activity may be presented. That is, the invention implemented in the form of each of the embodiments described above is also applicable to physical activities other than mountain climbing.

In each of the embodiments described above, the biological information detection section 221 detects the user US's blood oxygen concentration as the biological information. The invention does not, however, necessarily have the configuration. For example, the biological information detection section 221 may detect the user US's pulse wave, heart rate, blood sugar level, and other factors as well as the blood oxygen concentration. In this case, for example, the first threshold and the second threshold may be set on the basis of detected biological information as well as the blood oxygen concentration.

In the third embodiment described above, the warning information described above is transmitted only to the information processing apparatus 4B1 of the user US1, who is the leader. The destination of the warning information is not limited to the information processing apparatus 4B1, and the warning information may also be transmitted, for example, to the information processing apparatus 4B4 of the user US4, who is the subleader. Further, the information processing apparatus 4B1 to 4B4 of all the users US1 to US4 maybe connected to one another, and when any of the users US1 to US4 is in danger of suffering from altitude sickness, the warning screen WS3 described above may be displayed in the information processing apparatus of the other users out of the information processing apparatus 4B1 to 4B4.

In the third embodiment described above, the information processing apparatus 4B1 to 4B4 of the users US1 to US4 are connected to one another. The invention does not, however, necessarily have the configuration. For example, the measurement apparatus 2B1 to 2B4 of the users US1 to US4 may be connected to one another. In this case, the measurement apparatus and the information processing apparatus may have the same configurations as those of the measurement apparatus 2 and the information processing apparatus 4 according to the first embodiment.

For example, in a case where among the measurement apparatus 2B2 to 2B4, the measurement apparatus 2B1 of the user US1, who is the leader, is so set before mountain climbing that the warning information is transmitted thereto from the other measurement apparatus 2B2 to 2B4, when the control section 26 of any of the measurement apparatus 2B2 to 2B4 causes the breathing method presentation screen WS1 to be displayed in the display section 231 of the corresponding one of the measurement apparatus 2B2 to 2B4, the control section 26 transmits an instruction issued by the breathing instruction section 270 as the warning information to the measurement apparatus 2B1 of the user US1 via the communication section 24. The control section 26 of the measurement apparatus 2B1, when it acquires the warning information, causes the presentation information generation section 276 to generate the warning screen WS3 and causes the display section 231 of the measurement apparatus 2B1 to display the warning screen WS3.

As a result, also in the case where the measurement apparatus 2B1 to 2B4 are connected to one another, the same advantageous effects provided by the third embodiment described above can be provided.

In the third embodiment described above, the information processing apparatus 4B1 to 4B4 of the users US1 to US4 can be connected to one another. The invention does not, however, necessarily have the configuration. For example, the measurement apparatus 2B1 to 2B4 or the information processing apparatus 4B1 to 4B4 may be connected to another electronic apparatus over a network line. In this case, for example, when any of the users US1 to US4 has suffered from altitude sickness, the electronic apparatus can receive information on the occurrence of altitude sickness (warning information). As a result, since another person other than the users of the party who are performing mountain climbing can recognize the situation of the users US1 to US4, the party can be quickly rescued, for example, when three of the users US1 to US4 or the users US1 to US3 suffer from altitude sickness or any other problem.

In each of the embodiments described above, the control sections 26 and 450 read the notification control program stored in the storage sections 25 and 44 and execute the program. The invention does not, however, necessarily have the configuration. For example, the notification control program may be read from a recording medium and executed when the notification control process is carried out. Examples of the recording medium may include a magnetic tape, a magnetic disk, an optical disk, a magneto-optical disk, an HDD (hard disk drive), and a semiconductor memory. The notification control program can be not only installed in the information processing apparatus 4A and 4B (computer) and executed but also readily distributed by using any of the recording media described above. Further, the notification control program may be acquired from an apparatus on a network and executed.

What is claimed is:

1. A physical activity assistance apparatus comprising:
a blood oxygen concentration measurement section that measures a user's blood oxygen concentration; and
a breathing instruction section that instructs the user to practice a predetermined breathing method when the blood oxygen concentration is lower than or equal to a first threshold.

2. The physical activity assistance apparatus according to claim 1, further comprising:
a breathing detection section that detects the user's breathing;
a practice evaluation section that evaluates based on a result of the detection performed by the breathing detection section whether or not the user is practicing breathing according to the breathing method; and
a handling method instruction section that instructs the user to practice a handling method when the practice evaluation section determines that the user is practicing the breathing according to the breathing method and the measured blood oxygen concentration is lower than or equal to a second threshold lower than the first threshold.

3. The physical activity assistance apparatus according to claim 1, further comprising:
a breathing detection section that detects the user's breathing;
a practice evaluation section that evaluates based on a result of the detection performed by the breathing detection section whether or not the user is practicing breathing according to the breathing method;
a period acquisition section that acquires, when the practice evaluation section determines that the user is practicing the breathing according to the breathing method, a period for which the user has been practicing the breathing according to the breathing method; and
a handling method instruction section that instructs the user to practice a handling method when the acquired period is longer than a predetermined period and the measured blood oxygen concentration is not higher than a second threshold lower than the first threshold.

4. The physical activity assistance apparatus according to claim 2,
wherein the breathing detection section includes a first sensor that detects motion of the user's chest.

5. The physical activity assistance apparatus according to claim 4,
wherein the breathing detection section includes a second sensor that detects the user's body motion, and
the practice evaluation section evaluates based on results of the detection performed with the first sensor and the second sensor whether or not the user is practicing the breathing according to the breathing method.

6. The physical activity assistance apparatus according to claim 1, further comprising:
an altitude detection section that detects altitude; and
an adjustment section that adjusts the first threshold in accordance with the detected altitude.

7. The physical activity assistance apparatus according to claim 1, further comprising
a restriction section that restricts an instruction issued by the breathing instruction section when the blood oxygen concentration is higher than the first threshold.

8. A physical activity assistance system comprising:
the physical activity assistance apparatus according to claim 1; and
an information processing apparatus capable of communicating with the physical activity assistance apparatus,
wherein the physical activity assistance apparatus transmits a warning signal when the blood oxygen concentration is lower than or equal to the first threshold and the second threshold, and
the information processing apparatus includes a notification section that notifies the user of a warning when the warning signal is received.

9. A physical activity assistance system comprising:
the physical activity assistance apparatus according to claim 2; and
an information processing apparatus capable of communicating with the physical activity assistance apparatus,
wherein the physical activity assistance apparatus transmits a warning signal when the blood oxygen concentration is lower than or equal to the first threshold and the second threshold, and
the information processing apparatus includes a notification section that notifies the user of a warning when the warning signal is received.

10. A physical activity assistance method performed with use of an instruction apparatus that instructs a user to practice a breathing method, the method comprising:
acquiring the user's blood oxygen concentration; and
instructing the user to practice a predetermined breathing method when the blood oxygen concentration is lower than or equal to a first threshold.

11. The physical activity assistance method according to claim 10, further comprising:
detecting the user's breathing;
evaluating based on a result of the detection of the breathing whether or not the user is practicing breathing according to the breathing method; and
instructing the user to practice a handling method when a result of the evaluation shows that the user is practicing the breathing according to the breathing method and the measured blood oxygen concentration is lower than or equal to a second threshold lower than the first threshold.

12. The physical activity assistance method according to claim 10, further comprising:
detecting the user's breathing;
evaluating based on a result of the detection of the breathing whether or not the user is practicing breathing according to the breathing method;
when a result of the evaluation shows that the user is practicing the breathing according to the breathing method, acquiring a period for which the user has been practicing the breathing according to the breathing method; and
instructing the user to practice a handling method when the acquired period is longer than a predetermined period and the blood oxygen concentration is not higher than a second threshold lower than the first threshold.

13. The physical activity assistance method according to claim 10, further comprising:
detecting altitude; and
adjusting the first threshold in accordance with the detected altitude.

14. The physical activity assistance method according to claim 10, further comprising
restricting an instruction of the handling method when the blood oxygen concentration is higher than the first threshold.

15. A physical activity assistance program executed by a computer, the program causing the computer to perform;
acquiring a user's blood oxygen concentration; and
instructing the user to practice a predetermined breathing method when the blood oxygen concentration is lower than or equal to a first threshold.

16. A physical activity assistance system comprising:
a physical activity assistance apparatus including
a blood oxygen concentration measurement section that measures a user's blood oxygen concentration,
a first control section that generates warning information based on a result of analysis of the blood oxygen concentration, and
a first communication section that transmits the warning information; and
an information processing apparatus including
a second communication section that receives the warning information, and
a second control section that carries out a breathing method presentation process that instructs, based on the warning information, the user to practice a predetermined breathing method.

17. The physical activity assistance system according to claim 16,
wherein the first control section generates the warning information when the blood oxygen concentration is lower than or equal to a first threshold.

18. The physical activity assistance system according to claim 16,
wherein the physical activity assistance apparatus includes a first sensor that detects motion of the user' s chest.

19. The physical activity assistance system according to claim 17,
wherein the physical activity assistance apparatus includes an altitude detection section that detects altitude, and
the first control section adjusts the first threshold in accordance with the detected altitude.

20. The physical activity assistance system according to claim 17, further comprising
a restriction section that restricts an instruction issued by the breathing instruction section when the blood oxygen concentration is higher than the first threshold.
